(19) United States
(12) Patent Application Publication
(10) Pub. No.: US 2022/0317113 A1
(43) Pub. Date: Oct. 6, 2022

(54) METHOD FOR SCREENING THERAPEUTIC TARGET OF ACUTE GASTROINTESTINAL SYNDROME AND USE OF TIGAR TARGET IN PREPARATION OF MEDICINE FOR TREATING RADIATION-INDUCED GASTROINTESTINAL SYNDROME

(71) Applicant: SOOCHOW UNIVERSITY, Suzhou (CN)

(72) Inventors: Haowen ZHANG, Suzhou (CN); Chunlin SHAO, Suzhou (CN); Zhongmin WANG, Suzhou (CN); Yushuo ZHANG, Suzhou (CN); Xiaolin SHI, Suzhou (CN); Chen WANG, Suzhou (CN); Jinlu WANG, Suzhou (CN); Zicheng DENG, Suzhou (CN); Longxin LIN, Suzhou (CN); Fei CHEN, Suzhou (CN); Huangge REN, Suzhou (CN); Qimeng TANG, Suzhou (CN); Haoliang DUAN, Suzhou (CN); Bowen FU, Suzhou (CN); Fenju LIU, Suzhou (CN)

(21) Appl. No.: 17/427,617

(22) PCT Filed: Jun. 28, 2020
(86) PCT No.: PCT/CN2020/098434

§ 371 Date: Jul. 30, 2021

(30) Foreign Application Priority Data
Apr. 24, 2020 (CN) 202010334894.3
Apr. 24, 2020 (CN) 202010334901.X

(51) Int. Cl. G01N 33/50; A61K 38/17; A01K 67/027; C12N 15/85

(52) U.S. Cl. CPC G01N 33/5044; A61K 38/17; A01K 67/027; C12N 15/8509; C12N 2503/02; G01N 2500/10; A01K 2227/105; A01K 2267/03; A01K 2217/05

(57) ABSTRACT

The invention discloses a method for screening a therapeutic target of acute radiation-induced gastrointestinal syndrome and use of TIGAR target in the preparation of a medicine for treating radiation-induced gastrointestinal syndrome. The CreERT-loxP transgenic mouse model is used, in which quiescent intestinal crypt stem cells are effectively promoted to proliferate after exposure to high-dose ionizing radiation, to screen a therapeutic target that still has a therapeutic effect for radiation-induced gastrointestinal syndrome 18-24 h after ionizing radiation. Gene splicing occurs in particular cells in the CreERT-loxP transgenic mice only after the injection of tamoxifen, thereby regulating gene expression. The actual situation of initial exposure and then treatment after a nuclear accident is well simulated, so the invention is of great practical significance. The screened therapeutic target is developed into a medicine for treatment after nuclear accidents, to save precious time for the treatment after nuclear accidents.

### FIELD OF THE INVENTION

The present invention relates to the technical field of biomedicines, and more particularly to a method for screening a therapeutic target of acute radiation-induced gastrointestinal syndrome and use of TIGAR target in the preparation of a medicine for treating radiation-induced gastrointestinal syndrome.

### DESCRIPTION OF THE RELATED ART

With the development of nuclear industry and wide use of nuclear technology, the nuclear safety is becoming increasingly important. In the past decades, there have been many serious nuclear accidents. Both the accident at the Chernobyl nuclear power plant in the former Soviet Union and the accident at Fukushima nuclear power plant in Japan in 2011 caused uncontrollable release of radioactive materials, causing the surrounding people to expose to nuclear radiation. Moreover, unexpected nuclear terrorist attacks (such as “dirty bombs”) can also cause large numbers of people to expose to radioactive rays.

Different tissues of the human body have different degrees of damage after being exposed to radioactive rays. It is generally believed that the sensitivity of cells to radiation is positively correlated with the rate of cell proliferation, and negatively correlated with the degree of cell differentiation. Under physiological conditions, the intestinal epithelium of the human body is rapidly renewed as driven by the proliferation of intestinal stem cells. Because the stem cells in small intestinal crypts are in a state of rapid proliferation under physiological conditions, they are extremely susceptible to radiation-induced damage to lose their original ability of proliferation and division. The mitotic arrest of stem cells causes the intestinal epithelium to lose the source of cell renewal, resulting in serious damage to the integrity of the intestinal epithelium, breakage and shedding of intestinal villi, and loss of the original barrier and absorption functions. When the abdomen of mice is exposed to X-rays (or gamma rays) at a dose greater than 15 Gy, symptoms such as severe diarrhea, malabsorption of nutrients, weight loss and others caused by intestinal epithelial damage will occur in one week after exposure, and the mice will die of radiation-induced gastrointestinal syndrome caused by the above symptoms in ten days after exposure. It can be seen that under large-dose accidental exposure conditions, the human intestinal tissue is an important target tissue that suffers from radiation-induced damage.

The existing techniques related to the treatment of radiation-induced gastrointestinal syndrome mainly include:

1. Intraperitoneal injection of 3,3′-diindolylmethane (DIM) can improve the survival rate of mice irradiated at 13 Gy. However, the treatment effect is closely related to the time of administration after exposure. If administered within 2 h after exposure, the survival rate of mice is greater than 50%. However, if administered 24 h after exposure, the survival rate of mice is less than 30%, and the effect is undesirable. The reason is that 3,3′-diindolylmethane (DIM) improves the survival rate of intestinal stem cells mainly by promoting the repair of DNA damage, and the survival rate of stem cells can be improved only on condition that the DNA damage caused by ionizing radiation is successfully repaired within 1-2 h. When 3,3′-diindolylmethane (DIM) is administered 24 h after exposure, the DNA damage repair process of the cells has ended, and the apoptosis process is irreversibly initiated in the cells that are not successfully repaired. At this time, the drug cannot effectively reduce the intestinal cell death and intestinal epithelial breakdown, and thus the survival rate of exposed mice cannot be significantly improved. In addition, the radiation dose received by mice curable by intraperitoneal injection of 3,3′-diindolylmethane (DIM) is 13 Gy. For doses above 15 Gy, the protection effect is expected to be weaker than that with 13 Gy.

2. Hydrogen-rich water is orally administered to protect the intestinal flora, or a bioactive preparation such as intestinal flora transplantation is used to reduce the radiation-induced intestinal damage, or valeric acid in the metabolites of intestinal flora is used to combat the radiation-induced intestinal damage. The above-mentioned means of administration all direct at the intestinal micro-environment of flora, and lack the performance of targeting and the specificity for intestinal stem cells, thus having a slow onset of action. They are suitable for preventive administration before exposure, but not for post-exposure treatment. The therapeutic effect of administration after exposure is undesirable.

3. Traditional antioxidants. Some natural antioxidants and synthetic antioxidants, such as natural polyphenol compounds and selenium compounds, etc., have the effect of scavenging reactive oxygen species (ROS) and promoting DNA repair. However, the above compounds also lack the performance of targeting and the specificity for stem cells. Moreover, the antioxidants non-specifically scavenge destructive ROS and proliferation-related ROS signals, where the proliferation-related ROS signals are essential for promoting the proliferation of stem cells, and the scavenge of proliferation-related ROS inhibits the proliferation of intestinal stem cells to some extent. Therefore, due to the non-specific scavenge of proliferation-related ROS, the above-mentioned antioxidants cannot effectively promote the proliferation of intestinal crypt stem cells.

Although the preventive administration before exposure can reduce the damage of stem cells and the destruction of intestinal epithelium caused by radiation to a certain extent, nuclear accidents and terrorist attacks are usually unpredictable, and the existing post-exposure treatments are unlikely to reverse the irreversible death of intestinal stem cells caused by radiation (because the rapidly proliferating intestinal stem cells are particularly susceptible to ionizing radiation, and will irreversibly enter the apoptosis process within 6-12 h after exposure, upon which most drugs will even have no time to exert an effect in these sensitive cells). Therefore, patients with radiation-induced gastrointestinal syndrome cannot be effectively treated. Therefore, there is an urgent need to develop a new treatment strategy that is useful for post-exposure treatment.

With the development of precision medicine and the in-depth research of intestinal crypt stem cells in the scientific community, it has been found that in addition to the population of rapidly proliferating stem cells (Lgr5⁺ stem cells) in the intestinal crypts, there is also a small population of stem cells that are in a “quiescent” state under physiological conditions, that is, “quiescent crypt stem cells”. Such stem cells proliferate very slowly under physiological conditions and are not responsible for maintaining the renewal of intestinal epithelium. Due to their slow proliferation, they have relatively high radiation resistance. While massive death of Lgr5⁺ stem cells is caused by chemicals or ionizing radiation, these stem cells have the potential to proliferate and can replace Lgr5⁺ stem cells to proliferate and differentiate into intestinal villi epithelial cells to a certain extent. However, in the case of damage caused by high-dose ionizing radiation, the disintegration of intestinal epithelium often occurs within 3 days after exposure. The limited proliferation ability of quiescent crypt stem cells is not sufficient to reverse the destructed integrity of the intestinal epithelium in a short period of time, so the death of animals will still occur 7-10 days after exposure. Therefore, accelerating the proliferation of quiescent crypt stem cells in a short period of time after ionizing radiation is an extremely important treatment method. At present, there are no treatments for radiation-induced gastrointestinal syndrome with respect to quiescent crypt stem cells. There is an urgent need for basic research to provide reliable theoretical and experimental basis.

In medical research, research results based on animal experiments are usually closer to the actual situation in human body than in-vitro cell experiments, thus having greater scientific significance and practical value. However, considering the animal ethics and experimental cost, animal experiments are not suitable for large-scale drug screening (tens of thousands of drugs) and the screening of effective therapeutic targets. Instead, transgenic animal models targeting specific genes based on a certain theoretical basis are more pertinent and purposeful for disease treatment. The currently available transgenic animal models and other existing technologies related to the research of radiation-induced gastrointestinal syndrome mainly include:

1. Studies have found that p53 gene-dependent p53 upregulated modulator of apoptosis (PUMA) mediates the apoptosis of intestinal epithelial cells after radiation through the mitochondrial pathway. PUMA-deficient mice (ordinary knockout mice) show tolerance to high-dose ionizing radiation and have protection on Lgr5⁺ stem cells in the intestinal crypts. Due to the use of ordinary knockout mice, it is impossible for genetic intervention after the mice are exposed.

2. Studies have found that TLR3-deficient mice can also resist high-dose ionizing radiation that causes crypt cell death and intestinal damage. In terms of the mechanism of action, p53-dependent cell death releases intracellular RNA and mediates apoptosis through TLR3. This study suggests that the use of TLR3/dsRNA complex inhibitors has the potential to alleviate radiation-induced gastrointestinal syndrome. Similarly, due to the use of ordinary knockout mice, it is impossible for genetic intervention after the mice are exposed. As such, the effect of treatment of radiation-induced gastrointestinal syndrome by intervention on TLR3 after exposure cannot be predicted with the results of this study, and only the preventive effect of intervention on TLR3 before exposure on radiation-induced gastrointestinal syndrome can be predicted.

3. Research using knockout mouse model found that when the receptor absent in melanoma 2 (AIM2) of double-stranded deoxyribonucleic acid (dsDNA) damage is deficient in mice, the radiation-induced gastrointestinal syndrome can be effectively alleviated. The intestinal protection mechanism is that AIM2 can participate in the recruitment of and activate Caspase-1 and induce the pyrolysis of crypt stem cells. This process does not depend on the apoptosis signaling pathways related to Caspase-3 and Caspase-7. Similarly, due to the use of ordinary knockout mice, it is impossible for genetic intervention after the mice are exposed.

4. In 2019, a research team found that the over-expressed unconventional prefoldin RPB5 interactor (URI) protein can protect mice from gastrointestinal syndrome caused by radiation. Mice with normal URI expressions have a mortality of up to 70%. Completely knocking out the URI gene will cause the mice to die of radiation-induced gastrointestinal syndrome. The mechanism of protection by URI protein is that it mainly exists in the population of quiescent intestinal crypt stem cells, and the slower proliferation rate of this population prevents the mice from radiation-induced damage. However, when URI is knocked out, the β-catenin-c-MYC signaling pathway that is previously inhibited by URI is activated. The cells proliferate rapidly and are more susceptible to radiation-induced damage, which in turn leads to the death of mice from radiation-induced gastrointestinal syndrome. Although quiescent intestinal crypt stem cells are studied in this research, post-exposure genetic intervention is not performed, so the therapeutic effect against radiation cannot be predicted.

However, in ordinary knockout mice or overexpressed mice, the target gene is already stably knocked out or overexpressed, and the gene expression cannot be regulated after ionizing radiation. At present, the more advanced and sophisticated transgenic animal model is the CreERT-loxP transgenic mouse model. However, there is no related research on the use of CreERT-loxP transgenic mouse model in the treatment of radiation-induced intestinal damage.

### SUMMARY OF THE INVENTION

To solve the above problems, the present invention provides a method for screening a therapeutic target of acute radiation-induced gastrointestinal syndrome. The CreERT-loxP transgenic mouse model is used, in which quiescent intestinal crypt stem cells are effectively promoted to proliferate after exposure by high-dose ionizing radiation, to screen a therapeutic target that still has a therapeutic effect for radiation-induced gastrointestinal syndrome 18-24 h after exposure.

An object of the present invention is to provide a method for screening a therapeutic target of acute radiation-induced gastrointestinal syndrome, which comprises: exposing a CreERT-loxP transgenic mouse model having a candidate therapeutic target gene to ionizing radiation at 15-18 Gy, injecting an estrogen analog after exposure to induce the candidate therapeutic target gene to express, and screening a therapeutic target promoting the proliferation of quiescent intestinal crypt stem cells.

Preferably, the CreERT-loxP transgenic mouse model comprises a Bmi1-CreERT-loxP transgenic mouse model.

Preferably, the method specifically includes the following steps:

S1: inserting the candidate therapeutic target gene into the downstream of the loxP-STOP-loxP sequence, and inserting the constructed sequence into the mouse genome, to construct loxP mice with a candidate therapeutic target gene;

S2: co-breeding the loxP mice having the candidate therapeutic target gene with CreERT mice, screening the CreERT-loxP transgenic mice having the candidate therapeutic target gene for use as the mice in the experimental group, and using the loxP mice having the candidate therapeutic target gene as the mice in the control group;

S3: exposing the mice in the experimental group and the mice in the control group to high-dose ionizing radiation; and

S4: immediately after exposure, injecting an estrogen analog to induce the overexpression of the target gene, and screening a therapeutic target that promotes the proliferation of quiescent intestinal crypt stem cells by evaluating the therapeutic effect against radiation.

In the present invention, the mice in the control group and the mice in the experimental group need to be injected with tamoxifen. However, the mice in the control group lack recombinase, so even if injected with tamoxifen, gene splicing cannot be induced and the overexpression of therapeutic target gene cannot be induced.

Preferably, in Step S1, the constructed sequence is inserted into the H11 or ROSA26 locus of the mouse genome. In the present invention, the two gene loci mentioned above are commonly used loci for gene editing. Inserting a gene editing fragment at these two loci is unlikely to affect other existing genes.

Preferably, the dose of high-dose ionizing radiation is 15-18 Gy, the dose rate is 0.5-10 Gy/min, and the range of exposure is whole-abdomen exposure.

Preferably, the estrogen analog is tamoxifen.

Preferably, tamoxifen is injected at a dose of 4-5 mg/20 g body weight of mouse.

Preferably, the therapeutic effect against radiation is evaluated by the proliferation of intestinal crypt stem cells and the survival of mice.

Preferably, the proliferation of intestinal crypt stem cells is the proliferation of intestinal crypt stem cells 3-5 days after exposure.

Preferably, the survival rate of mice is the survival rate of mice in 30 days after exposure.

A second object of the present invention is to provide use of the TIGAR gene or protein in the preparation of a medicine for treating radiation-induced gastrointestinal syndrome.

Preferably, the medicine for treating radiation-induced gastrointestinal syndrome is a medicine promoting the proliferation of quiescent intestinal crypt stem cells.

Preferably, the medicine for treating radiation-induced gastrointestinal syndrome is TIGAR protein or a medicine for inducing the overexpression of TIGAR protein.

Preferably, the TIGAR protein is used to scavenge destructive ROS and retain the proliferation-related ROS signal in the quiescent intestinal crypt stem cells.

Preferably, the medicine for treating radiation-induced gastrointestinal syndrome is in a dosage form including injections, capsules, tablets, oral preparations or microcapsules.

Preferably, the radiation-induced gastrointestinal syndrome in human body is caused by large-dose ionizing radiation at a dose of 8-15 Gy.

Preferably, the medicine for treating radiation-induced gastrointestinal syndrome is administered within 24 h after large-dose ionizing radiation.

The present invention has the following beneficial effects.

In the present invention, the CreERT-loxP transgenic mouse model is used, in which quiescent intestinal crypt stem cells are effectively promoted to proliferate after exposure by high-dose ionizing radiation, to screen a therapeutic target that still has a therapeutic effect for radiation-induced gastrointestinal syndrome 18-24 h after exposure. Gene splicing occurs in particular cells in the CreERT-loxP transgenic mice only after the injection of tamoxifen, thereby regulating gene expression. By virtue of this characteristic, the actual situation of initial exposure and then treatment after a nuclear accident is well simulated in the present invention, so the present invention is of great practical significance. The screened therapeutic target is developed into a medicine for treatment after nuclear accidents, so as to save precious time for the treatment after nuclear accidents.

The present invention also provides the therapeutic target TIGAR protein screened by the above method. By overexpressing the TIGAR protein in the quiescent crypt stem cells after high-dose ionizing radiation, the proliferation-related ROS signal in the cells can be retained while the destructive ROS caused by radiation is scavenged, thereby effectively promoting the proliferation of quiescent intestinal crypt stem cells. By promoting the proliferation of quiescent crypt stem cells, the radiation-induced gastrointestinal syndrome caused by high-dose ionizing radiation can be treated. Even if the TIGAR protein expression in quiescent crypt stem cells is increased 24 h after a nuclear accident (24 h after radiation exposure), the radiation-induced gastrointestinal syndrome can still be effectively treated.

### BRIEF DESCRIPTION OF THE DRAWINGS

FIG. 1 shows the principle of the Cre-loxP transgenic animal model;

FIG. 2 shows a mouse model overexpressing TIGAR specific to quiescent crypt stem cells;

FIG. 3 shows the whole-abdomen exposure of mice;

FIG. 4 shows the overexpression of TIGAR in quiescent crypt stem cells induced after ionizing radiation;

FIG. 5 shows that TIGAR overexpression in quiescent intestinal crypt stem cells promotes the survival of exposed mice;

FIG. 6 shows that TIGAR overexpression in quiescent intestinal crypt stem cells promotes the reconstruction of intestinal crypts in exposed mice; and

FIG. 7 shows that TIGAR has a better ability to promote the proliferation of quiescent crypt stem cells than NAC.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

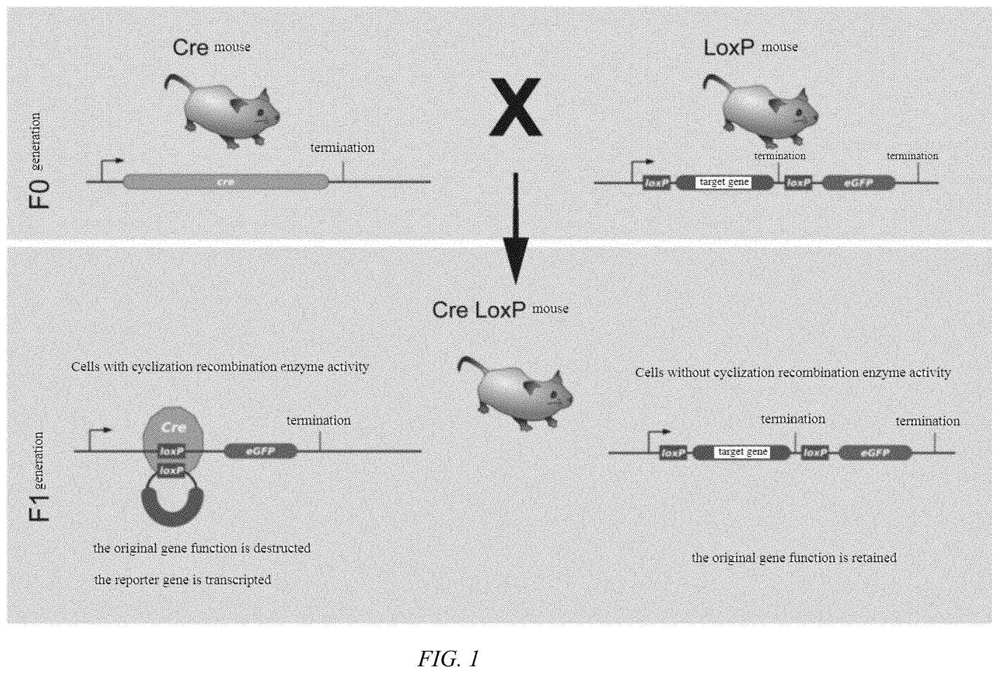

FIG. 1 shows the principle of the Cre-loxP transgenic animal model. In FIG. 1, the estrogen receptor (ER or ERT) that is closely related to the induction of expression is not indicated. When the Cre recombinase binds to the estrogen receptor, it cannot enter the nucleus to complete the cleavage. Only when a drug such as tamoxifen is injected, the Cre recombinase is unbound from the estrogen receptor, thereby completing the gene splicing. Only by using the above characteristic, gene induction and regulation can be achieved after ionizing radiation.

In the examples of the present invention, the Bmi1-CreERT;loxP transgenic mice are used as an example. The gene encoding the recombinase Cre is inserted in the downstream of a specific promoter (such as Bmi1) of quiescent intestinal crypt stem cells, to specifically regulate the gene in quiescent intestinal crypt stem cells, and simulate the treatment intervention after accidental exposure to the greatest extent in terms of temporal and spatial specificity, so as to effectively screen the genes that promote the proliferation of quiescent intestinal crypt stem cells after radiation damage.

### Example 1: Construction of CreERT-loxP Transgenic Mice

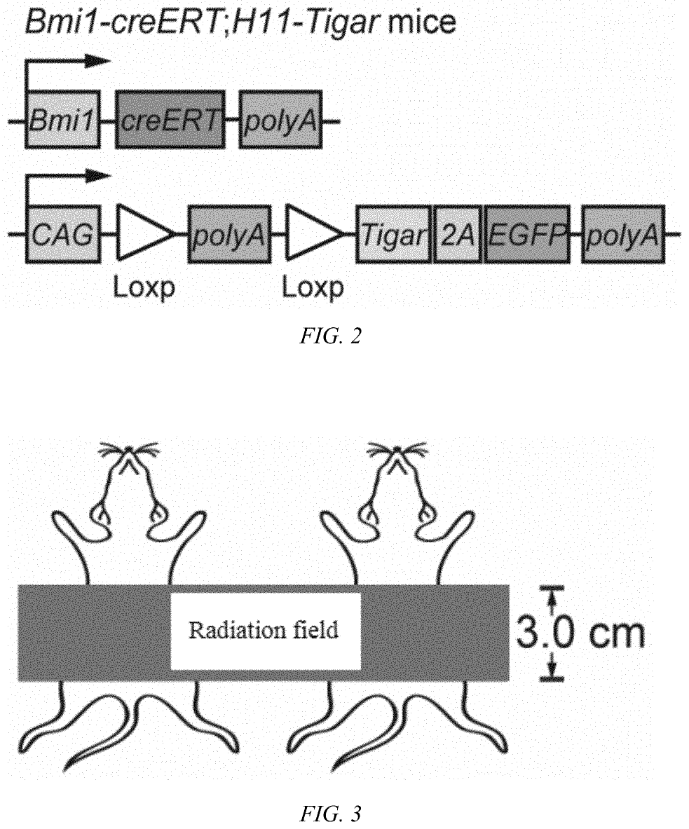

To effectively promote the proliferation of quiescent crypt stem cells, Bmi1-CreERT;loxP transgenic mice were used in this technical solution, and genetic intervention was performed on quiescent crypt stem cells in mice. TIGAR was used as a target gene, and TIGAR was induced to express in quiescent crypt stem cells by tamoxifen, as shown in FIG. 2.

In mice with the above-mentioned gene phenotype, TIGAR was allowed to be overexpressed only in quiescent crypt stem cells by Bmi1, a promoter specific to quiescent crypt stem cells.

The mice were designated as Bmi1-creERT;H11-Tigar. Specifically, Bmi1 is a specific promoter of quiescent crypt stem cells. Cre is a gene encoding recombinase, which can be translated into recombinase to cleave a specific gene sequence. ERT encodes the estrogen receptor. When creERT is translated as a whole, the recombinase binds to the estrogen receptor and cannot enter the nucleus to complete DNA splicing. Therefore, the estrogen receptor needs to be disassociated from the recombinase by injecting tamoxifen. After dissociation, the recombinase can enter the nucleus to splice a specific DNA sequence (loxP sequence). As a result of splicing, the gene sequence between two loxP sites is removed from the DNA sequence, and the remaining two truncated ends are spliced to form a new complete DNA sequence. For Bmi1-creERT;H11-Tigar mice, the result of splicing is that the STOP site (polyA) previously located upstream of Tigar is removed from the DNA sequence, and the Tigar gene that previously cannot be transcribed (due to the upstream STOP site) is allowed to be transcribed and translated after splicing, resulting in increased expression of Tigar protein. It takes 18-24 h from the intraperitoneal injection of tamoxifen to the achievement of overexpression of TIGAR protein.

The EGFP gene downstream of Tigar can be translated into green fluorescent protein to serve as a tracer. The 2A between Tigar and EGFP is a linker to ensure that TIGAR and EGFP will not fuse with each other after translation, causing the destruction of the spatial structure and loss of functions of the protein. Due to the presence of Bmi1, a specific promoter of quiescent intestinal crypt stem cells, the entire cleavage process mentioned above only occurs in quiescent intestinal crypt stem cells. In summary, by means of the above-mentioned animal model, the overexpression of TIGAR protein in quiescent crypt stem cells can be achieved 18-24 h after exposure. (tamoxifen is injected immediately after exposure)

Once the Tigar gene sequence is replaced by other genes with potential therapeutic value, the therapeutic targets of acute radiation-induced gastrointestinal syndrome can be screened. Of course, the above therapeutic target is proposed with respect to quiescent intestinal crypt stem cells.

### Example 2: Induction of Expression of Target Gene after Ionizing Radiation

Since it takes a certain period of time from drug injection to overexpression of TIGAR in quiescent crypt stem cells (usually 18-24 h for CreERT-loxP animal model), the drug was injected intraperitoneally (tamoxifen, single injection, 4.5 mg/20 g body weight of mouse) immediately after whole-abdomen exposure by X-rays at 15 Gy was received by the mice (FIG. 3).

On days 1, 3, and 5 after the mice were exposed, the mice were sacrificed and the intestinal tissues were made into frozen sections to observe the expression of TIGAR protein in quiescent crypt stem cells, as shown in FIG. 4. Since TIGAR and enhanced green fluorescent protein (EGFP) are expressed simultaneously during the design and construction of transgenic mice, the expression level of enhanced green fluorescent protein can be used to indicate the expression level of TIGAR. On day 1 after exposure, only 1-2 green cells are observed in the crypts, that is, quiescent crypt stem cells have been successfully overexpressed. Because quiescent crypt stem cells have the ability to divide and proliferate, a large number of progeny cells can be formed 3 and 5 days after exposure. The progeny cells of quiescent crypt stem cells also have green fluorescence, so the green fluorescence not only reflects the overexpression of TIGAR protein, but also reflect the progeny cells of quiescent crypt stem cells. The DAPI fluorescence in the figure is used to indicate the nucleus, facilitating better location and counting of the cells. It can be seen that 3-5 days after exposure, the reconstructed crypts consist essentially of the progeny cells of quiescent crypt stem cells, indicating that TIGAR overexpression promotes the proliferation of quiescent crypt stem cells and accelerates the reconstruction of crypts after exposure.

### Example 3: Evaluation of Therapeutic Effect Against Radiation

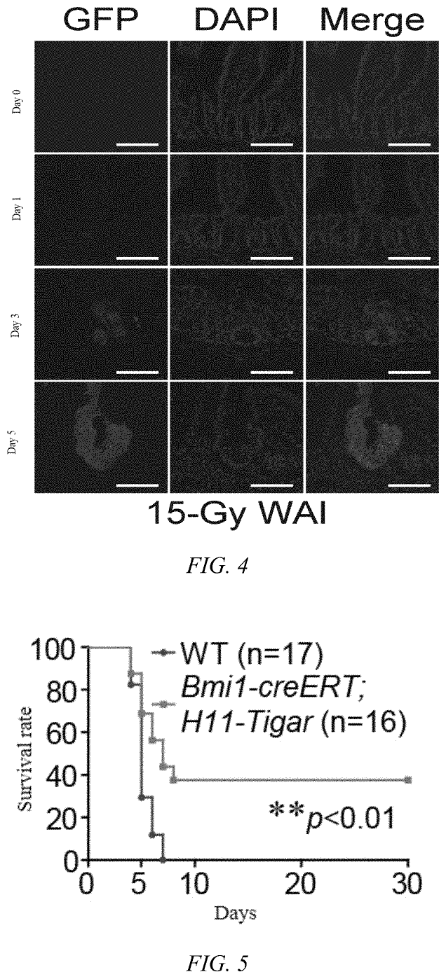

The therapeutic effect of TIGAR overexpression against radiation was evaluated by the survival rate of mice and HE staining of intestinal tissue sections. In the survival rate test, mice in the control group (where the Tigar gene was inserted downstream of the loxP-STOP-loxP sequence, to obtain the loxP-STOP-loxP-Tigar sequence, which was inserted into the H11 locus of the mouse genome to obtain H11-Tigar small mice) and mice with TIGAR overexpressed in quiescent intestinal crypt stem cells received whole-abdomen exposure by X-rays at 15 Gy (FIG. 3), and tamoxifen was injected intraperitoneally immediately after exposure (single injection, 4.5 mg/20 g body weight of mouse). After the injection, the mice were continuously bred to observe the survival of mice, as shown in FIG. 5.

It can be seen that mice in the control group (H11-Tigar mice, WT) all die of radiation-induced gastrointestinal syndrome (survival rate 0%) 7 days after exposure, and mice with TIGAR overexpressed in quiescent intestinal crypt stem cells (Bmi1-creERT;H11-Tigar) still have a survival rate of close to 40% 30 days after the exposure. Accordingly, the therapeutic effect is obvious (p<0.01).

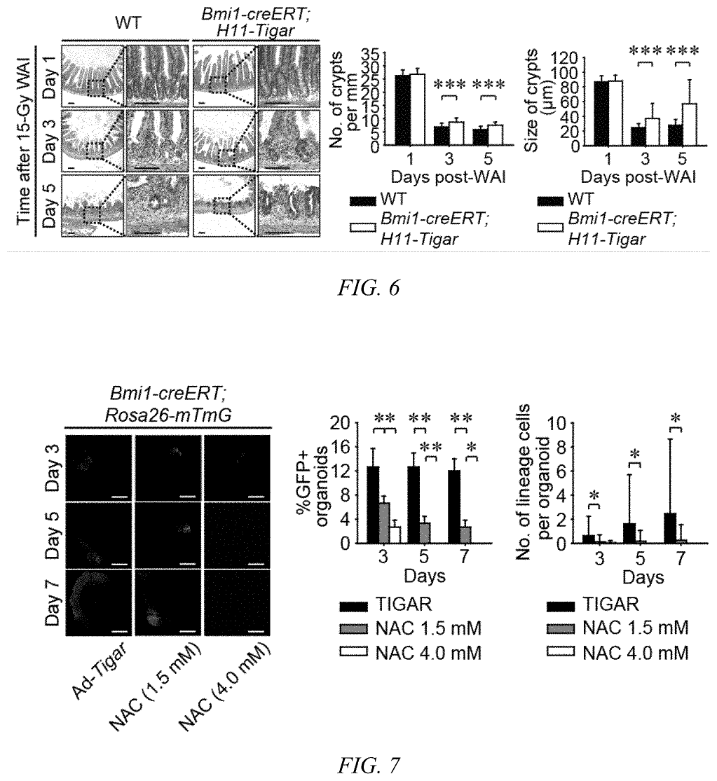

In the HE staining of intestinal tissue sections (as shown in FIG. 6), the mice were sacrificed 1, 3, and 5 days after exposure, and the intestinal tissues were collected and prepared into tissue sections for HE staining. It can be seen that with the proliferation of quiescent intestinal crypt stem cells after exposure, the number of intestinal crypts and the size of crypts in the mice with TIGAR overexpressed in quiescent intestinal crypt stem cells are significantly larger than those in the control group 3 and 5 days after exposure, suggesting that the TIGAR overexpression in quiescent intestinal crypt stem cells definitely promotes the proliferation and reconstruction of intestinal crypts after exposure. As the source of intestinal villi renewal, the timely reconstruction of intestinal crypts is of decisive significance for the treatment of radiation-induced gastrointestinal syndrome.

It is particularly to be noted here that although tamoxifen is injected intraperitoneally immediately after ionizing radiation in the experiment, TIGAR can only be considered to start to exert an effect 24 h after induction considering the time (18-24 h) needed for tamoxifen to induce TIGAR expression in quiescent intestinal crypt stem cells. Therefore, the idea mentioned in the object of the invention is confirmed that TIGAR can still promote the proliferation of intestinal crypt stem cells and promote the survival of mice 24 hrs after radiation exposure.

### Example 4: Evaluation of Effect on Promoting Proliferation of Quiescent Intestinal Crypt Stem Cells

To better reflect the effect of TIGAR on promoting the proliferation of quiescent crypt stem cells, the intestinal crypt organoid model cultured in vitro was used in the experiment. Intestinal crypt organoids were extracted from the small intestine of living mice. They had similar cell composition and proliferation kinetics to intestinal crypts in mice, and also had quiescent intestinal crypt stem cells.

The green fluorescence in Bmi1-creERT;Rosa26-mTmG mice was used to indicate the proliferation of quiescent intestinal crypt stem cells. The TIGAR was compared with the traditional reducing agent N-acetylcysteine (NAC) in promoting the proliferation of quiescent intestinal crypt stem cells, as shown in FIG. 7. It can be seen that TIGAR overexpression (transfected in adenovirus) can significantly increase the proliferation ability of quiescent crypt stem cells in the radiated crypt organoids, and the proliferation ability of quiescent crypt stem cells in the NAC treatment group is only promoted to a certain extent, which is, however, much lower than in the TIGAR treatment group (the number of green fluorescence-positive crypts and the count of green fluorescence-positive cells are significantly less than those in the TIGAR overexpressing group).

The traditional reducing agent NAC can remove both the destructive ROS and proliferation-related ROS signal in the cell, and TIGAR is reported to have the ability to scavenge only the destructive ROS, but retain the proliferation-related ROS. Therefore, the above experimental results show that TIGAR promotes the proliferation of quiescent intestinal crypt stem cells after exposure by specifically scavenging the destructive ROS and retaining the proliferation-related ROS in quiescent intestinal crypt stem cells.

The above-described embodiments are merely preferred embodiments for the purpose of fully illustrating the present invention, and the scope of the present invention is not limited thereto. Equivalent substitutions or modifications can be made by those skilled in the art based on the present invention, which are within the scope of the present invention as defined by the claims.

What is claimed is:

1. A method for screening a therapeutic target of acute radiation-induced gastrointestinal syndrome, comprising: exposing a CreERT-loxP transgenic mouse model having a candidate therapeutic target gene to ionizing radiation at 15-18 Gy, injecting an estrogen analog after ionizing radiation to induce the candidate therapeutic target gene to express, and screening a therapeutic target promoting the proliferation of quiescent intestinal crypt stem cells.

2. The method according to claim 1, wherein the CreERT-loxP transgenic mouse model comprises a Bmi1-CreERT-loxP transgenic mouse model.

3. The method according to claim 1, comprising specifically:
S1: inserting the candidate therapeutic target gene into the downstream of the loxP-STOP-loxP sequence, and inserting the constructed sequence into the mouse genome, to construct loxP mice with a candidate therapeutic target gene;
S2: co-breeding the loxP mice having the candidate therapeutic target gene with CreERT mice, screening the CreERT-loxP transgenic mice having the candidate therapeutic target gene for use as the mice in the experimental group, and using the loxP mice having the candidate therapeutic target gene as the mice in the control group;
S3: exposing the mice in the experimental group and the mice in the control group to ionizing radiation at 15-18 Gy; and
S4: immediately after ionizing radiation, injecting an estrogen analog to induce the overexpression of the target gene, and screening a therapeutic target that promotes the proliferation of quiescent intestinal crypt stem cells by evaluating the therapeutic effect against radiation.

4. The method according to claim 3, wherein in Step S1, the constructed sequence is inserted into the H11 or ROSA26 locus of the mouse genome.

5. The method according to claim 3, wherein the dose rate of the ionizing radiation is 0.5-10 Gy/min, and the range of exposure is whole-abdomen exposure.

6. The method according to claim 3, wherein the estrogen analog is tamoxifen.

7. The method according to claim 6, wherein tamoxifen is injected at a dose of 4-5 mg/20 g body weight of mouse.

8. The method according to claim 3, wherein the therapeutic effect against radiation is evaluated by the proliferation of quiescent intestinal crypt stem cells and the survival rate of mice.

9. The method according to claim 8, wherein the proliferation of quiescent intestinal crypt stem cells is the proliferation of quiescent intestinal crypt stem cells 3-5 days after ionizing radiation.

10. The method according to claim 8, wherein the survival rate of mice is the survival rate of mice in 30 days after ionizing radiation.

11. The method according to claim 1, wherein the therapeutic target promoting the proliferation of quiescent intestinal crypt stem cells comprises TIGAR gene or protein.

12. Use of the TIGAR gene or protein in the preparation of a medicine for treating radiation-induced gastrointestinal syndrome.

13. The use according to claim 12, wherein the medicine for treating radiation-induced gastrointestinal syndrome is a medicine promoting the proliferation of quiescent intestinal crypt stem cells.

14. The use according to claim 12, wherein the medicine for treating radiation-induced gastrointestinal syndrome is TIGAR protein or a medicine for inducing the overexpression of TIGAR protein.

15. The use according to claim 14, wherein the TIGAR protein is used to scavenge destructive ROS and retain the proliferation-related ROS signal in the quiescent intestinal crypt stem cells.

16. The use according to claim 12, wherein the medicine for treating radiation-induced gastrointestinal syndrome is in a dosage form of injections, capsules, tablets, oral preparations or microcapsules.
